SEQUENCE LISTING (8 sequences)

SEQ ID NO: 1 - protein, 36 aa, Artificial
  synthetic sequence
MGRSHHHHHHGMASMTGGQQMGRDLYDDDKDRWGSE

SEQ ID NO: 2 - protein, 7 aa, Artificial
  Xpress epitope
DLYDDDK

SEQ ID NO: 3 - DNA, 20 bp, Artificial
  Forward primer
atgagagggagcccatttgg

SEQ ID NO: 4 - DNA, 20 bp, Artificial
  reverse primer
ccgagatgtggaactggcag

SEQ ID NO: 5 - DNA, 24 bp, Artificial
  forward primer
tacatcagcacctctcaagcagag

SEQ ID NO: 6 - DNA, 20 bp, Artificial
  reverse primer
gaccagaatgtgccacggtt

SEQ ID NO: 7 - DNA, 25 bp, Artificial
  forward primer
catgctcaacatctcccccttctcc

SEQ ID NO: 8 - DNA, 25 bp, Artificial
  reverse primer
gggaaggtgtaatccgtctccacag

(19) United States
(12) Patent Application Publication
(10) Pub. No.: US 2012/0178177 A1
(43) Pub. Date: Jul. 12, 2012

(54) Biological Components Within the Cerebrospinal Fluid

(76) Inventors: Philippe Delerive, Courbevoie (FR); Zouher Majd, Ennetieres En Weppes (FR)

(21) Appl. No.: 13/390,172

(22) PCT Filed: Aug. 13, 2010
(86) PCT No.: PCT/IB2010/002201

§ 371 Date: Mar. 23, 2012

(30) Foreign Application Priority Data
Aug. 13, 2009 (EP) 09290628.8

(51) Int. Cl. G01N 21/64; G01N 33/68

(52) U.S. Cl. 436/172; 428/402

(57) ABSTRACT

The invention provides novel methods for isolating, characterizing, comparing, and using biological components that are present in the cerebrospinal fluid. Such biological structures, called CS-MPs, can be used for identifying biomarkers that reflect the status (or anticipate the development) of disorders of the Central nervous System (CNS). The novel methods, biological products, and related kits make possible the use of CS-MPs and of their components as biomarkers for the diagnosis, prognosis, or monitoring of CNS disorders. The CS-MPs have a diameter comprised between 100 and 1000 nm and contain phosphatidylserine (PS).

### TECHNICAL FIELD

The present invention relates to methods for isolating, characterizing, comparing and using specific biological components in the cerebrospinal fluid. Such biological components can be used for diagnosing or monitoring diseases in a subject, and/or for evaluating the therapeutic efficacy of a medical treatment or a candidate drug.

### BACKGROUND OF THE INVENTION

In recent years, huge progress has been made in the understanding of pathophysiology of the disorders of the Central Nervous System (CNS). However, the development of disease-modifying therapies remains a considerable challenge due to the absence of robust biomarkers for drug development, diagnosis, prognosis, and therapy. This aspect is particularly important for neurodegenerative disorders (such as multiple sclerosis and Parkinson's or Alzheimer's diseases) where validated biomarkers as surrogate endpoint for (pre)clinical drug development are needed (Shaw L et al., 2007; Dubois B et al., 2007; Pritchard J, 2008; Steinmetz K and Spack E, 2009).

Extensive studies have been performed in order to identify disease-associated and/or cell type-specific biomarkers using biological samples such as human or mouse brain tissues (Lyck L et al., 2008; Laterza O et al., 2006). In particular, Cerebrospinal Fluid (CSF) has been studied in recent years due to its close anatomical contact with brain interstitial fluid, and thus the simpler access to samples of diagnostic interest (de Jong D et al., 2007). Recent advances in biochemical analysis of human body fluids, such as CSF, have been applied to biomarker discovery and detection by using lipidomic, glycomic, proteomic and neuroimaging methods (Hu S et al., 2006; Fonteh A et al., 2006; Hwang H et al., 2010; Finehout E et al., 2007; Shi M et al., 2009; Hampel H et al., 2008).

CSF has a multiplicity of biological functions and a complex metabolism that can provide relevant information on CNS disorders (Johanson C et al., 2008). CSF volume and composition are the result of secretion and of other cellular functions that can be altered or fluctuate as a result of pathophysiological mechanisms, as shown by using compounds that regulate CSF formation or by studying CSF alterations due to pathologies and aging. Plasma components can be transported across the Blood-CSF Barrier (BCSFB or choroid plexus) and, through the Blood-Brain Barrier (BBB), the brain-CSF interface, and form the bulk of the CSF protein content (Roche S et al., 2008). Agents and diseases that affect BBB permeability and the cells forming the neurovascular unit may consequently alter CSF composition (Hawkins B and Davis T, 2005).

The protein fraction that mostly characterizes CSF, and probably the one having higher medical interest, is the result of CNS activity and/or disorders. In fact, different therapeutic or diagnostic strategies have been tested using CSF, which is now commonly considered as a promising source of biomarkers, in particular for neurodegenerative disorders such as Alzheimer's disease (Tumani H et al., 2008; Shaw L et al., 2009). These biomarkers can be proteins, or post-translationally modified variants, that are involved in CNS disorders such as Amyloid-beta, phospholipids and other biological elements (Visser P et al., 2009, Pasvogel A et al., 2008; de Jong D et al., 2007).

Complex datasets of proteins have been identified in the CSF by multi-dimensional chromatography and tandem mass spectrometry (Pan S et al., 2007; Huang J et al., 2007). CSF has been used for generating multianalyte profile with features for characterizing subjects that suffer from Alzheimer's disease or Parkinson's disease (Zhang J et al., 2008) or multiple sclerosis (Lehmensiek V et al., 2007), as well as for characterizing the difference in the CSF composition at the intra- and inter-individual level (Hu Y et at., 2005) or between human and rat CSF (Zappaterra M et al., 2007). A database listing the proteins identified in human body fluid proteomes, including CSF proteome, has been created (Li S et al., 2009).

However, all those studies encountered a common problem due to the presence of extremely abundant proteins coming from the blood (such as albumin, transferrin, acute phase proteins and various antibodies) that mask the more significant sub-proteome for identifying the biomarkers of medical interest that are generated within CNS or by other tissues in direct contact with the CSF (such as the cells forming BCSFB, BBB, or the brain-CSF interface). Even if specific analytical technologies are used to circumvent this problem (Zougman A et al. 2008; Thouvenot E et al., 2008), it remains difficult to distinguish the more variable but more difficult to separate, fraction of CSF proteome that is not derived from plasma (Roche S et al., 2008).

Means for the enhanced detection of CNS-released biologics would be useful for the medical management of CNS disorders, in particular for those that are characterized by inflammatory and/or degeneration processes in the CNS (or in CNS-associated tissues) that lead to cell type-specific apoptosis. Activation and apoptosis of neuronal cells have been shown in different in vitro models in which compounds such as Ceramide (Stoica B et al., 2005) or Staurosporine (Iwashita A et al., 2007) are used as stimuli. Moreover, proapoptotic and/or inflammatory effects of Lipopolysaccharides (LPS) and Staurosporine on CNS-associated cell types have been studied in vitro (Jung D et at., 2005; Kong G et al., 2002; Nagano T et al., 2006) and in vivo (Zujovic V et al., 2001; Sanna Petal., 1995).

During the last decade, preclinical and clinical studies led to the identification of circulating microparticles (MPs) as potential biomarkers of various biological functions or disorders that are associated to inflammation and apoptosis, as extensively reviewed (Burnier L et al., 2009; Doeuvre L et al., 2009). MPs are submicron fragments of the plasma membrane that are shed from cell membrane of apoptotic or activated cell types in response to various stress conditions and stimuli. MPs composition and membrane antigens may vary depending on their cellular origin and the type of stimulus involved in their formation. Other types of vesicular particles, called exosomes, are preformed vesicles that are released after cell activation, but diverge from MPs in size, surface antigens and clotting capacity (Horstman L et al., 2007; Thery C et al., 2009).

MPs are present in the blood of healthy individuals (being produced in particular by endothelial cells, platelets and other cells that circulate in the blood) but their absolute levels as well as the proportion of their different cellular origin may dramatically change under several pathological conditions. The presence and the activity of MPs have been extensively studied in plasma, as well as by generating and characterizing MPs in cell culture conditions, but some studies show that other biological fluids, such as urine or synovial fluids, present vesicle having features that are similar to those plasma MPs.

MPs formation is associated with the loss of membrane asymmetry and the exposure of specific phospholipids, such as phosphatidylserine, on the outer leaflet which, together with MPs surface antigen, are responsible of strong procoagulant activity that MPs normally exhibit. In fact, phospholipid-binding agents, in particular proteins have been used for the affinity-based isolation of MPs, after that they are separated from cellular components within plasma by centrifugation.

MPs are generally believed to have noxious properties, being capable of impairing endothelial activities, of increasing cytokine release, and of activating many other biological pathways. However, on the basis of more recent data on their specific composition and activities, MPs are now considered as a novel pathway to exchange information among cells through the activation of cell surface receptors or transporters that interact with circulating MPs (Morel O et al., 2009). Several studies tried to elucidate how the composition and/or the concentration of plasma or in vitro generated MPs can be associated to in vivo activities of potential therapeutic or diagnostic interest. Such qualitative and quantitative analyses of MPs were performed by using, in addition to classical flow cytometry analysis (Horstman L et al., 2007; Burnier L et al., 2009), proteomic technologies (Jin M et al., 2005; Garcia B et al., 2005). However, MPs have not been validated yet as biomarkers for any indication in clinical settings and improved means for characterizing MPs of medical interest are needed.

The possible association between MPs features (concentration and/or composition) and CNS pathologies has been suggested in literature but, as indicated in a recent review, new biological tools and methods are needed for identifying brain/neurovascular, tissue-specific MPs that can be used as reliable markers of CNS pathophysiological processes (Horstman L et al., 2007; Doeuvre L et al., 2009). The increase of MPs concentration into plasma has been described as being associated to diseases such as multiple sclerosis (Minagar A et al., 2001) or cerebral malaria, where MPs can alter BBB permeability (Faille D et al., 2009; Combes V et al., 2005). Procoagulant MPs have been detected in CSF and plasma after traumatic brain injury (Morel N et al., 2008) or in patients with acute basal ganglia hemorrhage (Huang M et al. 2009) but these authors have not provided any evidence on specific MPs populations that were originated by CNS.

In fact, all these data have been collected in conditions where the normal BBB structure and/or permeability are significantly disrupted (Hawkins B and Davis T, 2005). Thus, endothelial or blood-cells derived MPs can enter into CSF and, given the much lower concentration of proteins and particulate into CSF when compared to blood, can make the detection of CSF-borne MPs virtually impossible. In fact, no evidence is provided on if and how many MPs are actually generated by CNS, or by the other tissues in direct contact with the CNS or the CSF (such as the cells forming BCSFB, BBB, or the brain-CSF interface), and can be isolated from CSF.

Various cellular types that are present in the brain, such as the microglia and astrocytes, release MPs-like vesicle in response to the treatment with different compounds in cell culture conditions (Bianco F et al., 2005; Bianco F et al., 2009). Particles of cellular origin have been detected in samples of CSF of various origins but without identifying them on the basis of their CNS-specific as well as MPs-specific features that would allow defining them with sufficient precision and without confusing them with different entities such as exosomes (Huttner H et al. 2008; Bachy I et al., 2008; Marzesco A et al., 2005; Heegaard N et al., 2008; Scolding N et al., 1989). In absence of details such as the dimension, the presence of phosphatidylserine and the protocol for applying centrifugation to CSF samples (Horstman L et al., 2007), the findings in the literature on MPs-like elements that may be produced and shed in the CSF do not provide sufficient evidences in support of such a hypothesis (Smalheiser N, 2009). Thus there is a need for methods and products that allow the rapid and reliable detection of MPs-like elements into CSF samples, at the scope of identifying novel biomarkers for a number of CNS disorders, in particular neurodegenerative diseases without isolating samples of BBB or CNS tissues from individuals.

### SUMMARY OF INVENTION

The present invention relates to methods for isolating CNS-related, MPs-like elements into CSF samples (hereafter defined as Cerebrospinal Microparticles or CS-MPs). The methods involve the isolation of CSF samples (in particular of human, primate, or rodent origin) and separation of CS-MPs from the acellular fraction of CSF using phospholipid-binding agents (in particular phosphatidylserine-binding agents) and/or antigen-specific binding agents in a solid or a liquid phase.

The CS-MPs that are obtained by this method can be used to establish the concentration and/or components (such as cell type-specific antigens, phospholipids, or glycosylated groups) of CS-MPs that differ between control and test subjects (e.g., normal or at risk of a CNS disorder, treated or untreated for a CNS disorder). These molecular features can be used for defining biomarkers of medical interest for diagnosing or monitoring CNS disorders in a subject, and/or for evaluating the therapeutic efficacy of a medical treatment or a candidate drug.

Further objects of the present invention include kits and medical methods for diagnosing or monitoring a CNS disorder by isolating, characterizing, and using CS-MPs, as well further embodiments that are provided in the Detailed Description.

### DESCRIPTION OF THE FIGURES

FIG. 1—Characterization of features that differentiate CS-MPs populations.

An exemplary process for the characterization of features that characterize CS-MPs populations of different origin involves the isolation of Control CSF (e.g. from one or more healthy or untreated subjects) and of Test CSF (e.g. from one or more CNS disorder-affected or treated subjects) samples and the separation of the corresponding CS-MPs populations, which can be compared by any of the known methods for characterizing materials of biological origin. This analysis can allow determining specific features that are associated to CS-MPs populations in a category of subjects. This signature (i.e. biomarkers) can be used for evaluating the status of a subject with (or without) the isolation of CS-MPs populations.

FIG. 2—The characterization of feature(s) that differentiate CS-MPs from plasma MPs.

An exemplary process for the characterization of features that differentiate CS-MPs from Plasma MPs involves the isolation of CSF and of blood from one or more subjects and the isolation of the corresponding CS-MPs and Plasma MPs populations, which can be compared by any of the known methods for characterizing materials of biological origin. If this analysis is sufficiently validated in relevant populations of subjects, the normal (or CNS disorder-specific) features that are associated to CS-MPs populations can be determined when comparing CS-MPs to Plasma MPs in a category of subjects.

FIG. 3—Quantitative analysis of CS-MPs populations in rat CSF.

Two groups of male SD rats (n=8 for each group) were intracerebroventricularly injected with either vehicle (Saline; NaCl 0.9%) or a test substance (Lipopolysaccharides; LPS). After 24 hours, CSF was collected directly in the cisterna magna and blood was obtained by retro-orbital puncture from each animal. CS-MPs and plasma MPs populations were prepared as described in the Materials & Methods of Example 1 and the concentrations of CS-MPs (A) and Plasma MPs (B) populations in the samples were quantified for both groups of animals by flow cytometry. Data are expressed as Mean±SEM (*: p<0.05). Similar results were obtained in three independent experiments. In a control experiment (C), the CSF and blood samples were collected respectively in the cisterna magna and by retro-orbital, puncture from healthy male SD rats (n=8). CS-MPs and Plasma MPs populations were prepared as described in the Materials & Methods section of Example 1 and then quantified by flow cytometry. Data are expressed as Mean±SEM (*: p<0.05).

FIG. 4—Characterization of feature(s) that allow distinguishing CS-MPs (sub) populations that have specific cellular origins.

An exemplary process for the characterization of feature(s) that allow differentiating CS-MPs (sub) populations involves the generation and isolation of MPs from specific primary cells or cell lines that are related to CNS and to CSF metabolism (e.g. neural cells, glial cells, or cells associated to blood-brain barrier). The MPs populations that are generated and isolated from control and test cells (e.g. differentiated or undifferentiated, healthy or in an apoptotic state), can be compared by any of the known methods for characterizing materials of biological origin. If this analysis is sufficiently validated in relevant cell types, then these features (such as cell surface antigens) can be applied to the analysis of CS-MPs populations and their components for determining if any of them can be defined as a biomarker associated to normal (or CNS disorder-specific) CS-MPs populations in different categories of subjects.

FIG. 5—Isolation of test and control MPs from a cell line of neuronal origin.

The SH-SY5Y cells were maintained and differentiated in cell culture conditions as described under the Materials & Methods section of Example 2. Cells were then treated for 24 hours with Staurosporine at two different concentrations or with vehicle only. At the end of the treatment, culture medium was collected and MPs were isolated and subsequently quantified by flow cytometry. Those results are representative of several independent experiments. The resulting MPs populations have been isolated and characterized by flow cytometry (A) or by two-dimensional gel electrophoresis (B) as described in the Materials and Methods section. The three gels have been compared in an area comprised between approximately 10-60 kDa and pl 3.0 to 7.0. The boxes (named C, 1, and 2) define specific areas of interest in the captured images.

FIG. 6—Molecules known as potential CNS or BBB biomarkers that can be associated to specific CS-MPs subpopulations

The names and the relevance of selected molecules known as CNS or BBB biomarkers that have been identified in the literature as being associated to the CSF and/or the tissues associated to CSF (and that can be studied using specific CS-MPs subpopulations) are listed. Molecules such CD45, GFAP, NCAM variants, ALCAM and transporter proteins at the BBB are those of major interest, given their cell specificity and biological activities.

FIG. 7—Quantitative analysis of CS-MPs populations in human CSF.

The CS-MPs populations have been isolated from human CSF obtained from ten subjects, nine of them grouped as Control subjects. A statistically relevant increase of CS-MPs populations was detected in the test subject when compared to the control subjects. This result suggests that the more advanced state of the CNS disorders in the Test subject can be associated to the shedding of MPs from cells localized in the CNS, such MPs being detected as CS-MPs.

### DETAILED DESCRIPTION OF THE INVENTION

The present invention relates to methods for isolating the biological components in the CSF, hereafter defined as CS-MPs. The CS-MPs that are isolated by this method can be used for obtaining information that is potentially relevant for medical scope, in particular for defining and comparing biomarkers that are associated to CNS disorders.

The term “CS-MPs” refers to phospholipid-containing vesicles of cellular origin that have a dimension comprised between 100 and 1000 nanometers, that are present in the CSF, and that are originated by cell types that form CNS, including the brain (e.g. neuronal cells, glial cells), the spinal cord, as well as those forming the tissues that regulate the exchange of molecules between brain and blood (i.e. the cells that form the BBB, the BCSFB, and the brain-CSF interface). The content of phospholipids can be determined using a phosphatidylserine-binding agent such as Annexin V (Genebank NM_{—}001154) or Lactadherin (Lemmon M, 2008; Stace C and Ktistakis N, 2006), or using enzymatic assays that measure procoagulant phospholipids (Van Dreden P et al., 2009; Osumi K et al., 2001; Huang M et al., 2009).

The Examples show that, in addition to the presence of phosphatidylserine in CS-MPs, the origin of the CS-MPs populations can be established to be associated to the desired cell type on the basis of the presence of specific antigens, in particular those localized on the cell surface, that are known from the literature or further identified by analyzing MPs generated by cell lines or primary cells.

The term “biomarker” refers to an entity that is an objectively measured factor predicting or signaling a specific state of an organism (including the predisposition, the progression, and the improvement of a CNS disorder). In the present case, this factor can be defined by the concentration and/or the components of CS-MPs that are isolated from the CSF of humans or animals (rodents or primates, in particular). The biomarker can be found associated to the whole CS-MPs population and/or to specific CS-MPs subpopulations defined by any molecular parameter of interest (for example, the presence of a further cell type-specific antigen or amount of phospholipids). Thus, the quantitative evaluation of CS-MPs (sub)populations (to be provided as the number of CS-MPs present in specific volume of CSF) can be, or not, associated to a quantitative evaluation of CS-MPs, such as the ratio, the concentration of CS-MPs presenting (in particular on the surface) such component of interest. Thus a biomarker can be a cell component (e. g. a protein, a protein variant, a cell-specific antigen, a phospholipid, a nucleic acid, a glycosylated group) or any other organic or inorganic elements that can be found associated to an CS-MPs population (e.g. a virus, a drug, an antibody, and any other compound that may interact with the surface of CS-MPs).

The term “CNS disorder” includes any disorders affecting the brain, the spinal cord and the tissues more physically and functionally associated such as the BBB, the BCSFB, and the brain-CSF interface. A CNS disorder can be an acute or chronic disease that involves the pathological disruption, inflammation, degeneration, and/or proliferation of the cells forming the brain, the spinal cord, the BBB, the BCSFB, or the brain-CSF interface. An association between a biomarker (such as a CS-MPs population or a CS-MPs component) and a CNS disorder can be established independently from the cause of the disorder, by applying the statistical analysis to biological samples of potential relevance, such as the CSF and plasma, and/or other clinical parameters.

A non-exhaustive list of CNS disorders includes Parkinson's disease, Parkinson syndrome, Alzheimer's disease, Down's disease, amyotrophic lateral sclerosis, familial amyotrophic lateral sclerosis, progressive supranuclear palsy, Huntington's disease, spinocerebellar ataxia, dentatorubral-pallidoluysian atrophy, neuropathies, olivopontocerebellar atrophy, cortical basal degeneration, familial dementia, frontal temporal dementia, senile dementia, diffuse Lewy body disease, striatonigral degeneration, chorea athetosis, dystonia, Meigs's syndrome, late cortical cerebellar atrophy, familial spastic paraplegia, motor neuron disease, Machado-Joseph disease, Pick's disease, nervous dysfunction after cerebral apoplexy, nervous dysfunction after spinal damage, demyelinating disease (for example, multiple sclerosis, Guillain-Barre syndrome, acute disseminated encephalomyelitis, acute cerebellitis, transverse myelitis, etc.), brain tumor (for example, astrocytoma, neuroblastoma, meningioma, medulloblastoma, CNS lymphoma, and other neuroepithelial/neuroectodermal tumors), cerebrospinal disease caused by viral or bacterial infections (for example, meningitis, cerebral abscess, AIDS, etc.), mental disease (schizophrenia, manic-depressive psychosis, neurosis, psychosomatic disease, epilepsy, etc.) and the like.

The CS-MPs can allow the identification of biomarkers for characterizing the state of a subject (such as normal, affected or at a risk of disorder, responding or not to a therapy) by using samples obtained from such subject. The biological samples can be the CSF as such, or the CS-MPs population isolated from the CSF, or even plasma. In fact, if the BBB or the BCSFB permeability or structure is altered, the CS-MPs populations that are characterized in the CSF may pass in the blood and then also detected in this biological fluid.

A method for identifying CS-MPs comprises the following steps:

a) Obtaining a CSF sample from a subject;

b) Isolating the acellular fraction of said CSF sample;

c) Separating the CS-MPs from the said acellular fraction by means of their dimension and the presence of at least one molecule that is known to be associated to cells forming the brain, the spinal cord, the BBB, the BCSFB, or the brain-CSF interface;

wherein the CS-MPs have a diameter comprised between 100 and 1000 nanometers and contain phosphatidylserine.

The method involves the isolation of CSF samples from humans, primates, rodents, or any other animal presenting an interest for medical or veterinary research. The CSF samples can be obtained by puncture, involving the removal of a volume of at least 0.01 ml (e.g. in rodents and smaller animals) up to 1 or more ml (e.g. in human or primates). It is of major importance to ascertain that the CSF sample is not mixed with blood that results from the rupture of arterial or venous walls during the puncture, thus excluding any contamination of the CS-MPs populations in the CSF sample by plasma MPs that, for instance, present typical platelet antigens such as CD42.

The isolation of the acellular fraction of the CSF is performed by eliminating any cellular elements having a size superior to 1000 nanometers, as it is possible by flow cytometry, microfiltration, or centrifugation. Consistently with the literature, the centrifugation of the CSF samples at a speed comprised preferably between 1,500 g and 15,000 g, at a temperature comprised between 15° C. and 37° C., and for a time comprised between 1 minutes and 60 minutes should allow the separation of fraction containing the CS-MPs (the supernatants) from the cells (forming the pellet).

As shown in the Examples, two centrifugation steps are generally applied, a first one at low speed (below 5,000 g) for eliminating the majority of cells as a pellet, and then this first supernatant is centrifuged at higher speed (between 10,000 g and 15,000 g) to generate the acellular fraction of the CSF to be used for isolating CS-MPs populations. Alternatively, a second centrifugation of the CSF samples at a speed comprised between 15,000 g and 50,000 g (and more preferably between 20,000 g and 40,000 g), at a temperature comprised between 15° C. and 37° C., and for a time comprised between 1 minutes and 60 minutes should allow the separation of fraction containing the CS-MPs (forming the pellets) from the other, lighter elements present in the CSF in the supernatant.

The separation of the CS-MPs from the acellular fraction of a CSF sample can be performed by applying technologies for isolating cell vesicles having a diameter comprised between 100 and 1000 nanometers, as well as a by detecting a component that is typical of CS-MPs, such as phosphatidylserine, and/or a molecule that is known to be associated to cells forming the brain (e.g. neuronal cells and/or glial cells), the spinal cord, the Blood-Brain Barrier (BBB), the Blood-CSF Barrier (BCSFB), or the brain-CSF interface. These criteria (i.e. the dimension and the presence of phosphatidylserine) can be applied in any order and, for the latter one, agents having affinity for molecules that are known to be associated to cells forming the brain, the spinal cord, the BBB, the BCSFB, or the brain-CSF interface should be also used for obtaining the desired CS-MPs populations. In particular a phospholipid-binding agent and an agent having affinity for molecules that are known to be associated to neuronal cells and/or glial cells can be used for separating the CS-MPs from the acellular fraction of the CSF sample.

The phrase “agent having affinity” refers to any material that can bind to the desired molecule (that is, a component of CS-MPs such a protein, a protein variant, a phospholipid, a nucleic acid, a glycosylated group, etc.) and consequently allow detecting and/or separating the structures containing such molecule (i.e. CS-MPs) in a sample (i.e. the acellular fraction of a CSF sample), preferably by interacting with components on the surface of CS-MPs. The agent having affinity for the desired molecule can be a natural or recombinant protein (such as an antibody or a protein that binds a cell surface antigen), a peptide, an inorganic compound, a nanomaterial, a nucleic acid, etc. The agent having affinity for molecules is a phospholipid-binding agent or an agent that binds an antigen associated to cells forming the brain (e.g. neuronal cells and/or glial cells), the spinal cord, the BBB, the BCSFB, or the brain-CSF interface

The agent having affinity for the desired molecule (that is, a component of CS-MPs) can be labeled. There are numerous methods by which the label can produce a signal detectable by external means, for example, desirably by visual examination or by electromagnetic radiation, heat, and chemical reagents. The label or other signal producing system component can also be bound to a specific binding partner, another molecule or to a support. The label can directly produce a signal, and therefore, additional components are not required to produce a signal. Numerous organic molecules, for example fluorescers, are able to absorb ultraviolet and visible light. Other labels that directly produce a signal include radioactive isotopes and dyes. Alternately, the label may need other components to produce a signal, and the signal producing system would then include all the components required to produce a measurable signal, which may include substrates, coenzymes, metal ions, substances that react with enzymatic products, etc.

Moreover, the agent having affinity for the desired molecule can be provided in a liquid phase or in a solid phase (for example, by the immobilization on a bead or a plate from which it can be separated or not), forming thus a complex with the CS-MPs once that the acellular fraction of a CSF sample is contacted with such agent. Subsequently, depending on the further uses, such complex can be dissociated (for instance, by temperature or chemical-induced denaturation) or the agent having affinity for the desired molecule can be kept associated to the CS-MPs.

The use of phospholipid-binding agents, and in particular of phosphatidylserine-binding agents (Lemmon M, 2008), as the agent having affinity is of particular importance, given that MPs have been often isolated by that means in a solid or a liquid phase. Additional agents having affinity to be used according to the invention can be defined as agents that bind a relevant antigen, such as the ones cited in the literature as being associated to cells forming the brain, the spinal cord, the BBB, the BCSFB, or the brain-CSF interface (FIG. 6). Alternatively, CS-MPs populations can be indirectly detected on the basis of an in vitro or in vivo assay, such as the procoagulant activity that is well established for plasma MPs (Van Dreden P et al., 2009; Osumi K et al., 2001; Huang M et al., 2009).

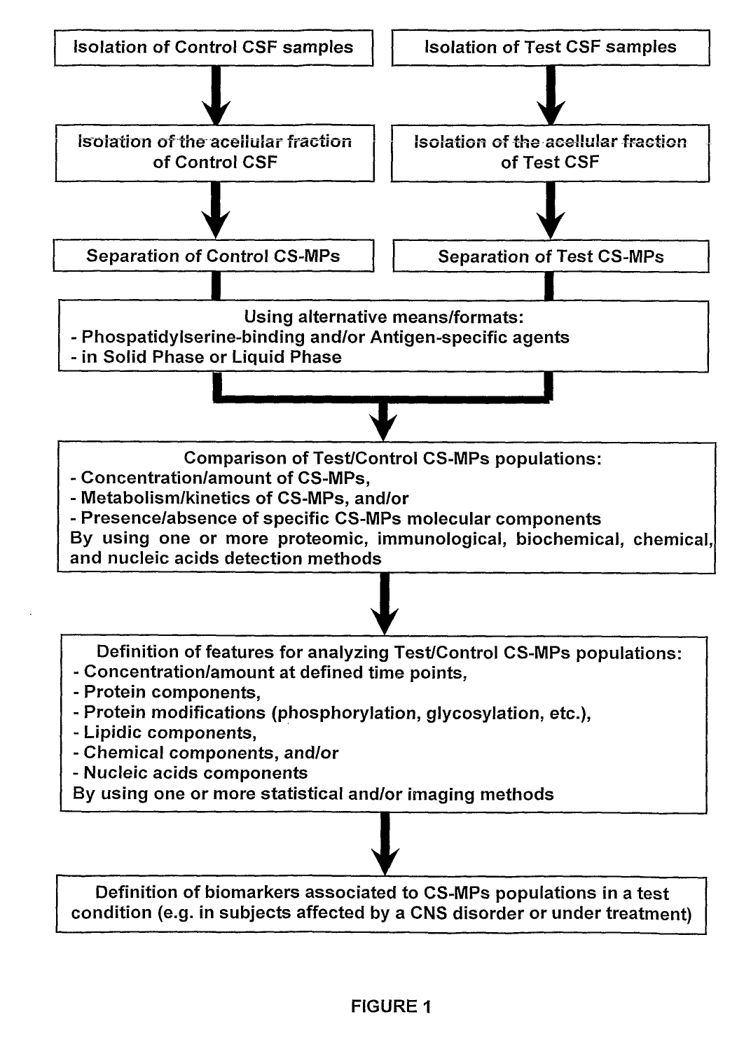

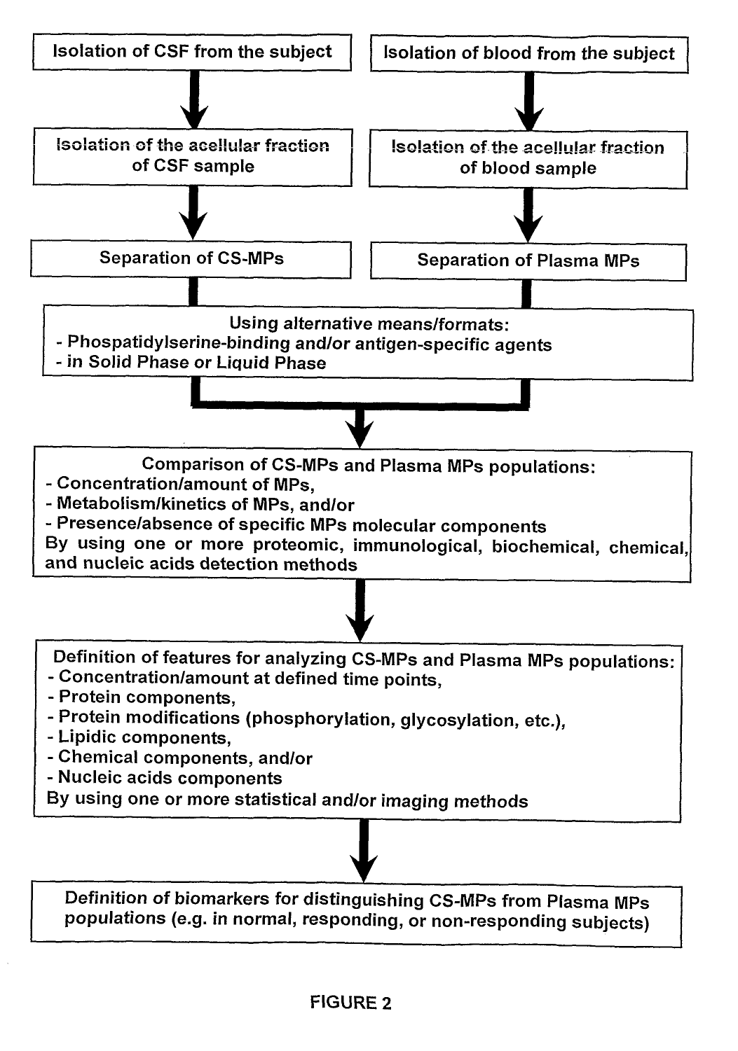

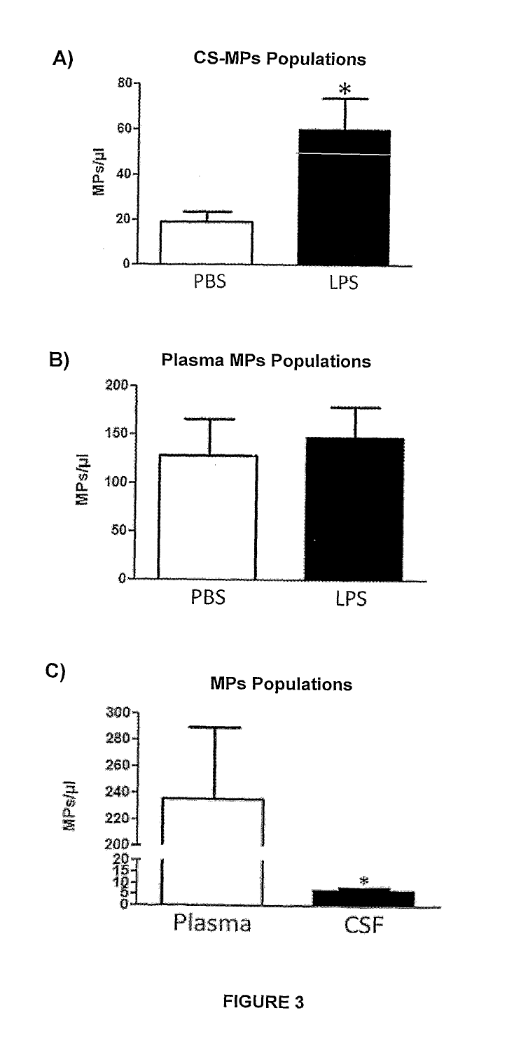

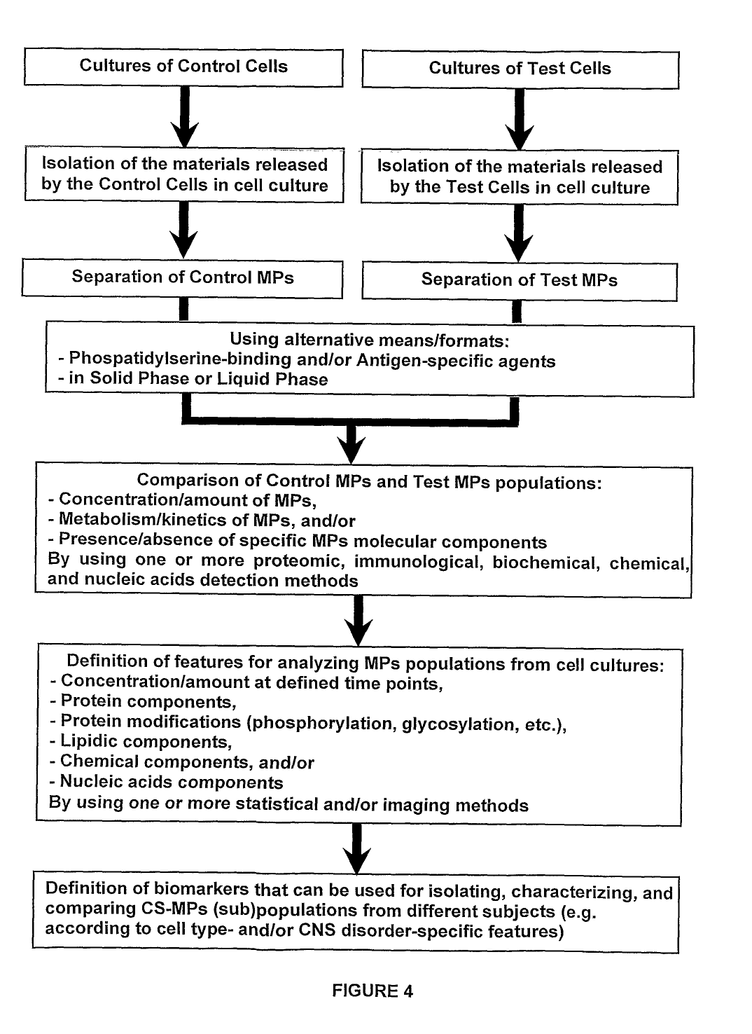

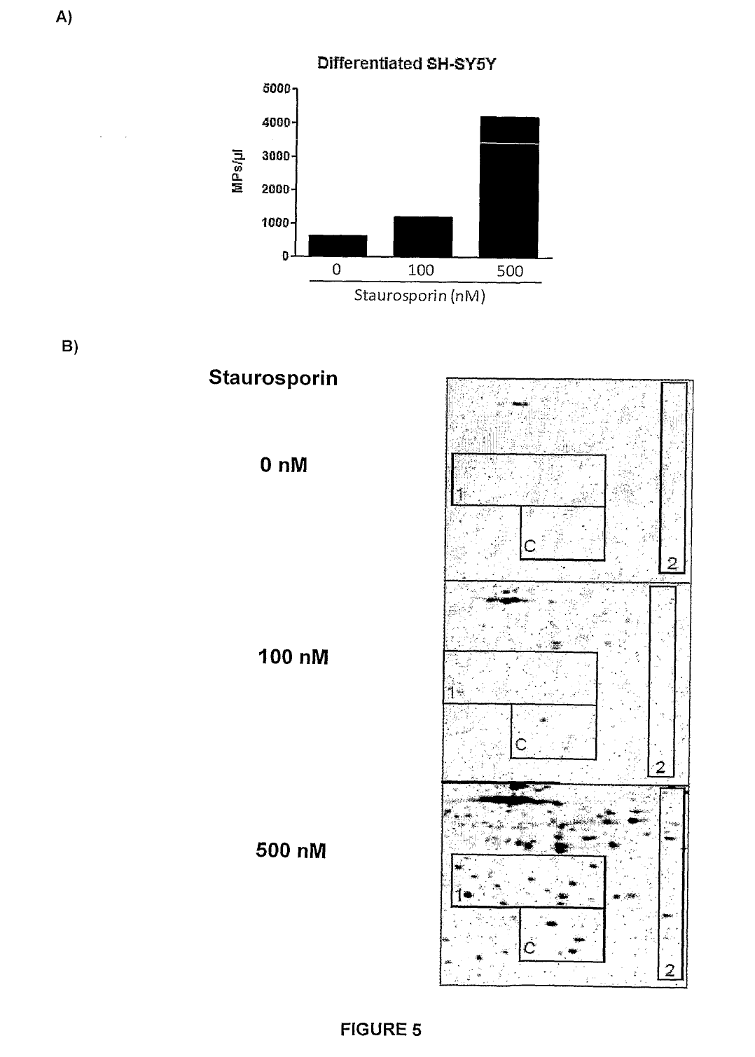

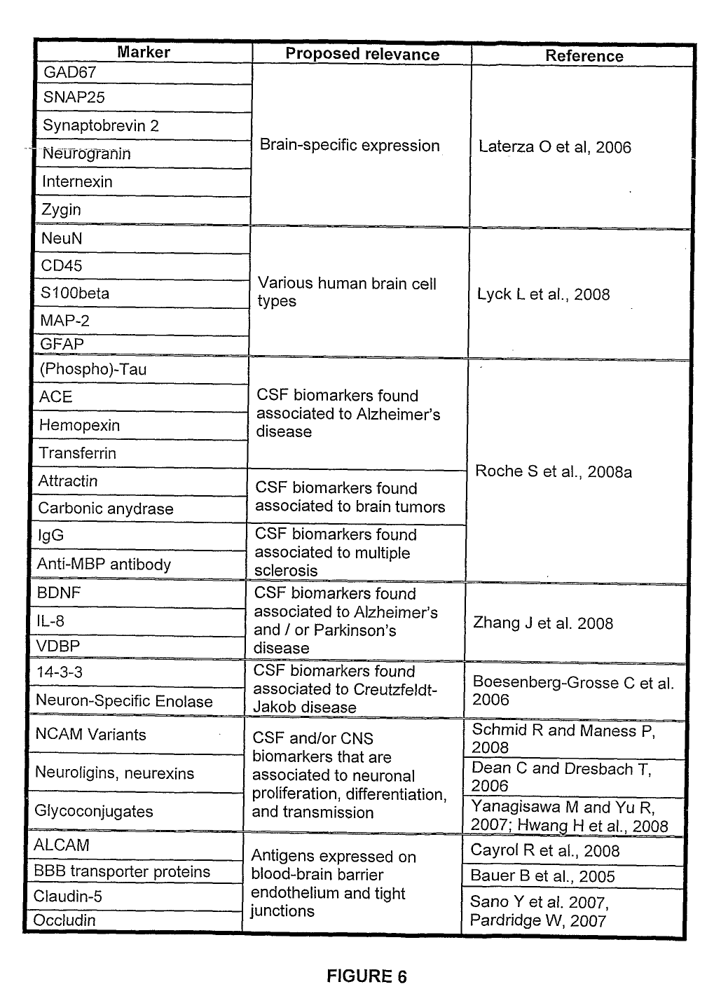

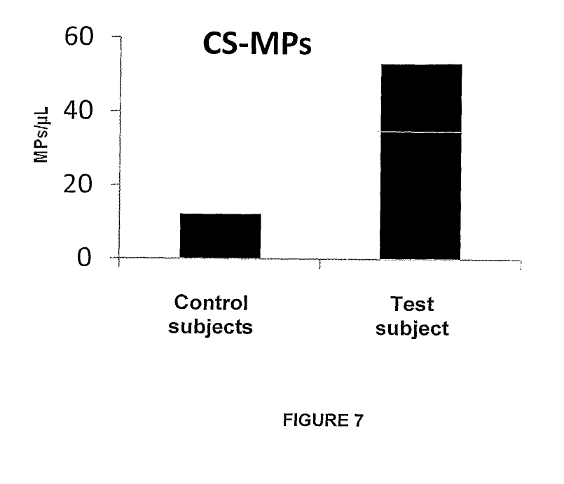

The methods of the invention provide novel biological entities that are defined, separated, and obtained as CS-MPs. The CS-MPs that can be provided in a liquid or a solid phase, and in association or not with the agent having affinity for molecules that are known to be associated to cells forming the brain, the spinal cord, the BBB, the BCSFB, or the brain-CSF interface. These novel biological entities are defined in connection to specific Central Nervous System (CNS) cell types and/or disorders and then can provide novel biomarkers that are associated to CS-MPs in general or to specific CS-MPs subpopulations of interest that are defined in connection to specific Central Nervous System (CNS) cell types and/or disorders. For instance, such biomarker can be used for isolating CS-MPs populations and/or for screening subjects at risk of being affected by a CNS disorder, by using common technologies such as flow cytometry, mass spectrometry, gel electrophoresis, an immunoassay (e.g. immunoblot, immunoprecipitation, ELISA), nucleic acid amplification, procoagulant activity, and/or electron microscopy on CSF samples or CS-MPs populations obtained from such subjects in a singleplex or multiplex formats (FIGS. 1 and 2).

The present Invention also relates to the kits for isolating and/or using CS-MPs for medical or veterinary application. Such kits comprise at least a phospholipid-binding agent and an agent having affinity for the desired molecule (e.g. a monoclonal antibody against a component of CS-MPs surface) that can be provided in a liquid or a solid phase, as well as means for detecting and comparing effectively the phosphatidylserine-containing CS-MPs (and consequently for quantifying the CS-MPs (sub)population of interest) by using one or more proteomic, immunological, biochemical, chemical, biological, or nucleic acid detection method.

The CS-MPs of the invention can be isolated, selected, characterized, compared, and used according to desired medical application. Examples of the process for analyzing and comparing CS-MPs populations and identifying biomarkers of medical interest are summarized in FIGS. 1 and 2, but many other possibilities can be envisaged in connection to specific medical goals, features of the biomarker, and/or the type of subjects to be evaluated. In particular, the CSF samples into which CS-MPs features are studied, can be obtained from distinct groups of subjects that are appropriately selected (e. g. on the basis of drug treatment, age, sex, pathologies, genotype, phenotype, exposure to risk factors, viral infection, clinical status, etc.) and then compared at the level of CS-MPs (sub)populations using biomarkers that can be evaluated by means of one or more proteomic, immunological, biochemical, chemical, biological, or nucleic acid detection method.

The outcome of this comparison, that may also involve the use of appropriate statistical and/or imaging methods, may lead to the confirmation or the identification of a biomarker associated to a CS-MPs (sub)population that can be further used in diagnostic, drug discovery, and drug validation methods for a CNS disorder, as well as of any other disorder that may alter the structure and/or the activity of the cells in the brain (e.g. neuronal cells and/or glial cells) and the spinal cord, as well as of those forming the tissues that regulate the exchange of molecules between brain and blood (i.e. the cells that form the BBB, the BCSFB, and the brain-CSF interface).

The present Invention also relates to the medical methods that involve the isolation, the characterization, and the comparison of CS-MPs populations. In fact, although the diagnostic use of MPs within CSF has been suggested, methods for a specific and efficient identification, characterization, and comparison of CS-MPs populations have not been disclosed so far. As numerous plasma proteins that are now considered as relevant biomarkers of various clinical conditions, quantitative and/or qualitative features of CS-MPs populations can be of considerable value for diagnosing and monitoring of CNS disorders, as well as for evaluating drug candidates and drug treatments for any disease, and in particular for establishing their effects on CNS, CSF, and/or the barriers separating them from blood.

Such methods make use of the CS-MPs populations that have been obtained by the methods of the invention for diagnosing or monitoring of a disorder, such a CNS disorder, altering the composition of CSF in general, and of CS-MPs concentration and/or composition more particularly, in a sample as it can be determined by flow cytometry, mass spectrometry, gel electrophoresis, an immunoassay (e.g. immunoblot, immunoprecipitation, ELISA), nucleic acid amplification, procoagulant activity, and/or electron microscopy on CSF samples or CS-MPs populations (that is, by applying technologies that allow the identification of biomarkers of interest).

The term “diagnosing” refers to diagnosis, prognosis, monitoring a disorder in a subject individual that either has not previously had the disorder or that has had the disease but who was treated and is believed to be cured. This application of the methods of the invention can be extended to the selection of participants in (pre) clinical trials, and to the identification of patients most likely to respond to a particular treatment.

The term “monitoring” refers to tests performed on patients known to have a disorder for the purpose of measuring its progress or for measuring the response of a patient to a therapeutic or prophylactic treatment, and in general for evaluating the therapeutic efficacy of a medical treatment or a candidate drug.

The term “treatment” refers to therapy, prevention and prophylaxis of a disorder, in particular by the administration of medicine or the performance of medical procedures with respect to a patent, for either prophylaxis (prevention) or to cure the infirmity or malady in the instance where the patient is afflicted.

The approaches that are summarized in FIGS. 1, 2, and 4 allow the definition of novel biomarkers in form of novel molecular features that are associated to CS-MPs populations, and consequently to the CSF, as a result of CNS biological functions and complex metabolism (Johanson C et al., 2008). Once that biomarkers are found associated to CS-MPs, such biomarkers can be identified in the subjects of interest (e.g. animal models, patients, at risk individuals) for obtaining information of medical interest on a subject, throughout the time (e.g. before and after a medical intervention or treatment) and/or in comparison to specific reference populations (e.g. control, healthy subjects or subjects affected by a disorder).

The invention provides novel method for diagnosing or monitoring a CNS disorder in a subject by determining the concentration and/or the composition of CS-MPs, in particular by detecting a biomarker associated to CS-MPs (sub) populations. These methods may involve the isolation of CS-MPs within the CSF, but may involve the identification of CS-MPs within other biological fluids (e.g. blood, urine) where they can pass and can be isolated by means of a biomarker of interest. Optionally these methods may also involve comparing the concentration and/or composition of total MPs, or of other specific MPs populations, that are present in another biological fluid of the subject (for example Plasma MPs). Still optionally, the methods may involve the detection of the biomarker(s) found associated with CS-MPs, within a tissue. Such tissues can be the ones from which CS-MPs can be originated (e.g. obtained from biopsies of the CNS) but can also be any other cell types or biological material of interest for diagnosing or monitoring a disorder. The methods of the invention can thus provide a biomarker profile that combines the amount of protein or as amount of gene transcript, of a number of biomarkers specific for diagnosing or monitoring CNS disorders that are associated to CS-MPs, within CSF, as well as in other biological samples.

It will be understood that it is not absolutely essential that an actual control sample be run at the same time that assays are being performed on a test sample. Once “normal” (i.e. control) concentration and/or composition of CS-MPs (i.e. CS-MPs vs plasma MPs ratios, CS-MPs presenting or not a specific cell surface antigen) have been established, these levels can provide a basis for comparison without the need to rerun a new control sample with each assay. The comparison between the test and control samples using appropriate statistical methods and criteria should provide a basis for a conclusion on the state of the subject, for instance whether the disorder is progressing or regressing in response to a treatment or if the subject is affected or not by a disorder, or if the control sample is contaminated by CSF (being CS-MPs an indirect indication of the presence of CSF) as consequence of the rupture of BCSFB or any other traumatic event.

All references cited herein are fully incorporated by reference in their entirety. Having now fully described the invention, it will be understood by those of skill in the art that the invention may be practiced within a wide and equivalent range of conditions, parameters and the like, without affecting the spirit or scope of the invention or any embodiment thereof.

### EXAMPLES

### Example 1

### Quantification of Rat CS-MPs Compared to Plasma MPs

### Materials & Methods

### Isolation of Rat CSF

Male Sprague-Dawley rats (225-250 g, CERJ, France) were anesthetized with pentobarbital (55 mg/kg) and positioned in a stereotaxic frame. The rat head was flexed downward at approximately 45 degrees, a depressible surface with the appearance of a rhomb between occipital protuberances and the spine of the atlas becomes visible. The 25 G needle was punctured into the cisterna magna for CSF collection without making any incision at this region. The blunt end of the needle was inserted into a 10 in. length of PE-50 tubing and other end of the tubing was connected to a collection syringe (Hamilton, 100 μl). The non-blood contaminated sample (100 μl) was drawn into the syringe by simple aspiration. Samples with blood cell contaminations were discarded. A sample was centrifuged at 13,000 g for 2 minutes and the resulting supernatant was subsequently snap-frozen in polypropylene tubes. Samples were stored at −80° C. until analysis.

### Isolation of Rat Platelet-Free Plasma

Blood was collected by retro-orbital puncture from Male Sprague-Dawley rats (225-250 g, CERJ, France) and transferred into a tube containing 1/10 volume of citrate buffer in order to prepare platelet-rich plasma. Within one hour of collection, platelet-rich plasma was obtained by centrifugation at 1,500 g for 15 minutes at room temperature. The supernatant is then carefully removed and transferred to a new tube. Platelet-free plasma is then obtained by centrifugation at 13,000 g for 2 minutes at room temperature. Again, the supernatant is carefully transferred into a new tube and snap-frozen using liquid nitrogen. Samples were stored at −80° C. until use.

### Production and Purification of Recombinant Human Annexin V Protein

The DNA encoding human Annexin V (Genebank NM_{—}001154) was used for producing Histidine-tagged, recombinant Annexin V in bacteria (E. Coli strain BL21 star P10S). The recombinant protein results from the fusion of the DNA sequence coding for a synthetic sequence (MGRSHHHHHHGMASMTGGQQMGRDLYDDDKDRWGSE; SEQ ID NO: 1) that includes an hexahistidine tag and the Xpress epitope (DLYDDDK; SEQ ID NO: 2; Invitrogen Life Technologies), in 5′ to the DNA encoding human Annexin V (amino acids 1-320). The Histidine-tagged, recombinant Annexin V was purified using an HIS-Trap column (GE Healthcare). Purity was assessed on gel and sequence was verified by mass spectrometry. His-tagged Annexin V was then labelled with NHS-Fluorescein (Thermo-Scientific, Pierce Protein Research Products; Cat. No. 46410) or fluorescein isothiocyanate (FITC), following the manufacturer's protocol.

### Quantification of Rat CS-MPs and Plasma MPs

The quantifications were performed using a FC500 flow cytometer (Beckman-Coulter, France) by incubating for 30 minutes in the dark each samples (30 μl) with 60 ng of His-tagged, Fluorescein-labelled Annexin V in incubation buffer (2.5 mM CaCl₂, 140 mM NaCl, 10 mM HEPES pH 7.4). In addition, fluorescent calibrated beads (Flowcount Fluorospheres, Beckman-Coulter, France; v/v, beads/sample with a 0.5 μm cut-off to obtain reproducible results) were added to each sample in order to express CS-MPs counts as absolute number per μl of sample. Each sample was then analyzed by flow cytometry.

### Intracerebroventricular Injection of Rats

This experiment was elaborated on the basis of protocols described in previous publications (Sanna P et al., 1995). Briefly, male SD rats (225-250 g, CERJ, France) were anesthetized with pentobarbital (55 mg/kg) and positioned in a stereotaxic frame. Then, they received an intracerebroventricular injection of LPS (50 μg/animal in 3.3 μl of saline buffer) or saline (NaCl 0.9%) using a Kopf apparatus. The coordinates for the sterotaxic injections were −2.3 mm dorsal/ventral, −1.4 mm lateral, and −0.6 mm anterior/posterior from the bregma.

### Gene Expression Analysis

Total RNA was extracted using Qiagen RNA extraction kits following manufacturer's instructions. Total RNA was treated with DNase I (Ambion Inc., Austin, Tex., USA) at 37° C. for 30 minutes, followed by inactivation at 75° C. for 5 minutes. Real time quantitative PCR (RT-QPCR) assays were performed using an Applied Biosystems 7900 sequence detector. Total RNA (1 μg) was reverse transcribed with random hexamers using Taqman reverse-transcription reagents kit (Applied Biosystems) following the manufacturer's protocol. Gene expression levels were determined by Sybr green assays. 36B4 transcript was used as an internal control to normalize the variations for RNA amounts. Gene expression levels are expressed relative to 36B4 mRNA levels.

The specific primers used for the quantification were:

(SEQ ID NO: 3)
- For TNFalpha, Forward sequence is ATGAGAGGGAGCCCATTTGG
and
(SEQ ID NO: 4)
reverse sequence is CCGAGATGTGGAACTGGCAG;
(SEQ ID NO: 5)
- For IL1beta, Forward sequence TACATCAGCACCTCTCAAGCAGAG
and
(SEQ ID NO: 6)
Reverse sequence is GACCAGAATGTGCCACGGTT;
(SEQ ID NO: 7)
- For 36B4, Forward sequence is CATGCTCAACATCTCCCCCTTCTCC
and
(SEQ ID NO: 8)
Reverse sequence is GGGAAGGTGTAATCCGTCTCCACAG.

### Results & Conclusions

The molecular features of potential medical interest that constitute CS-MPs can be characterized as biomarkers by applying an approach in which CSF samples of different origin are isolated and compared for the quantitative and qualitative features using appropriate technologies (FIG. 1). Initially, the CS-MPs populations should be obtained and defined by using a process that allow comparing their concentration and cellular origin, in particular by distinguishing the contribution of MPs already present into plasma from MPs that are actually originated from tissues in direct contact with CSF, to total CS-MPs. This approach can be extended to a more general evaluation on the molecular features that differentiate CS-MPs from Plasma MPs population in the same subjects from the quantitative, metabolic, and/or molecular point of view (FIG. 2).

A series of experiments were designed to quantify in parallel CS-MPs and Plasma MPs in different conditions using rat models. The quantification of rat CS-MPs compared to Plasma MPs has been performed by positively staining Annexin V-binding MPs that are identified in rat CSF and plasma samples. The analysis was performed in samples wherein the cellular elements have been eliminated by centrifugation, and restricted to particles having a diameter comprised between 100 and 1000 nanometers. Then, in order to determine if CS-MPs number could be affected by an acute stress in the brain, a test compound (LPS) was injected intracerebroventricularly in the rat brain to mimic a neuroinflammatory state. As a control, the expression of a number of inflammatory markers such as IL1alpha and TNFalpha was monitored at the gene expression level in various areas of the brain including the cortex, striatum and the hippocampus. It has been observed that LPS injection in the CNS leads to a huge increase in the expression of selected inflammatory markers (40-fold and 6-fold induction for IL1beta and TNFalpha, respectively).

The intracerebroventricular injection of LPS resulted also in a significant 3-fold induction of CS-MP levels (FIG. 3A), without a simultaneous and significant increase of Plasma MPs (FIG. 3B). Moreover, when compared to Plasma MPs (232±147 MPs/μl), CS-MPs levels are low but detectable (7±1 MPs/μl ) even in absence of a stress, such as the injection, that may cause CS-MPs release (FIG. 3C).These data suggests that CNS disorders and/or related treatments can induce the increase (or the decrease) of CS-MPs concentration independently from the plasma MP levels. Since CS-MPs that are originated from the CNS can be identified within CSF, the additional CS-MPs that are induced by a stimulus (such as a compound providing an apoptotic or neuroinflammatory effect, a drug, or a neurotoxic compound) can provide biomarkers on the effect of such stimulus on CNS activities that are detected in the CSF.

Thus CS-MPs populations that are isolated from CSF can be used for the evaluation of treatments, predispositions, and/or progression related to CNS disorders involving such tissues, as described above with a compound mimicking neurotoxicity and/or neuroinflammation. Similar experiments can be performed in toxin-based animal models using other compounds such as 1-methyl-4-phenyl-1,2,3,6-tetrahydropyridine (MPTP), whose injection may lead to neuronal loss and inflammation, two key events occurring during the development of Parkinson's disease (Jenner P, 2008). Alternatively, this method could be used to monitor the CNS response to the alteration in the expression of specific genes, in animal models, for example in transgenic mice or using RNA interference technologies for the local delivery of small interfering RNAs, miRNAs or shRNAs that are carried by lentiviral or adenoviral vectors (Rohl T and Kurreck J, 2006).

### Example 2

### Quantification of MPs Produced in Cell Culture Conditions

### Materials & Methods

### Cell Culture Protocols

The human neuronal cell line SH-SY5Y (ATCC CRL-2266) was cultured in MEM/Ham's F12 medium (1:1) supplemented with 10% foetal bovine serum. Cells were seeded at 55,000 cells/cm². Cell differentiation was induced by adding 9-cis retinoic acid (5 μM) for 5 days directly to complete culture medium. After 5 days, medium was replaced with complete medium supplemented with BDNF (50 ng/ml) for additional 5 days. At the end of the differentiation protocol, cells were rinsed with serum-free medium and then treated for 24 hours with complete medium containing Staurosporine (100 and 500 nM; Sigma, France) or vehicle (DMSO 0.1%).

### MPs Quantification

At the end of the treatment, cell supernatants were collected and spun down for 15 minutes at 1,500 g at room temperature. MPs were then isolated by ultracentrifugation at 30,000 g for 45 minutes at room temperature. At this speed, both exosomes and ectosomes are not sedimented (Thery C et al., 2009). Supernatants were discarded and pellets were re-suspended in PBS buffer. Samples were stored at −80° C. until use.

MPs quantifications were performed by flow cytometry using fluorescein-labelled, His-tagged Annexin V, as indicated in Example 1 for CS-MPs and Plasma MPs.

### Two-Dimensional Gel Electrophoresis

MPs populations were obtained from Staurosporine-induced SH-SY5Y cells as indicated above. Following the step of ultracentrifugation, total MPs populations were used for preparing a protein extract into 0.5 ml of 2D lysis buffer (7M urea, 2M thiourea, 4% CHAPS, 20 mM spermine base, 0.8% DTT, 0.2% pH 3-10 BioLytes, phosphatase inhibitor cocktails 1 & 2, and EDTA-free protease inhibitor cocktail tablets). The proteins were fully extracted on ice by sonication with 20 second pulses at 20-25% of maximum amplitude level. This step was repeated 5 times with a minimum of one minute pauses. Extracts were centrifuged at 16,100 g for 10 minutes at 10° C. to remove insoluble particles. Non-protein impurities were removed using the ReadyPrep 2D Cleanup kit prior to isoelectrofocalisation (IEF) steps. Protein pellets were solubilized by incubating them with 450 μl of rehydration buffer (7M urea, 2M thiourea, 4% CHAPS, 0.4% DTT and 0.2% pH 3-10 BioLytes) at room temperature for 5 minutes.

After a further centrifugation at 16,100 g for 5 minutes at 22° C. to remove insoluble fragments, samples were subjected to IEF using premade 24 cm IPG strips, nonlinear pH 3-10, on an IPGphor instrument (Bio-Rad Laboratories, Hercules, Calif., USA) in electrophoresis buffer (7M urea, 2M thiourea, 4% CHAPS, 0.4% DTT and 0.2% pH 3-10 BioLytes) at 20° C. for a total of 140 kVh achieved with a maximum tension of 8000V. After a low current 500V final step, strips were refocused at 8000V for 30 minutes.

Prior to the second dimension, proteins in strips were reduced and alkylated by using 10 mL of equilibration buffer (6M urea, 2% SDS, 50 mM Tris-HCl pH 8.8, 20% glycerol, 2% DTT for 15 min and then for 15 minutes with the same buffer without DTT and containing 2.5% iodoacetamide). Equilibrated IPG strips were shortly soaked in 0.22 μm-filtered electrophoresis buffer and immediately transferred onto 20.2×25.5 cm in size and 1 mm thick gradient 8-16% polyacrylamide gradient gels (38:1 acrylamide:bis ratio) pre-casted into low-fluorescence glass plates treated with bind and repel silane and including 2 fluorescent markers (Jule Inc, CT, USA). Strips were immobilized by embedding them into a 0.5% agarose solution in electrophoresis buffer that was labeled with trace amounts of bromophenol blue. The second dimension separation was performed overnight at 30° C. using an Ettan Dalt six device with an updated upper buffer chamber (GE Healthcare) according to the manufacturer's instructions.

After electrophoresis, gels were fixed and then stained for total proteins by an overnight incubation in SYPRO Ruby solution (Invitrogen, Carlsbad, Calif., USA). After extensive washings, image acquisition was performed using a Typhoon 9400 laser scanner (GE Healthcare) at a 100 μm resolution using the 457 nanometers excitation wavelength.

### Results & Conclusions

Since the composition of CSF depends, at least in part, from the drainage of the interstitial liquid of the nervous tissues in the brain, CSF (and consequently CS-MPs) may be therefore considered as a reporter of the pathophysiological status of the CNS. This possibility can be confirmed by identifying CNS-specific markers into CS-MPs populations or, even better, markers that are associated to specific cell types present in CNS and associated to different CNS activities. The molecular features that allow differentiating cell type-specific CS-MPs subpopulations of potential major interest may be initially defined by means of specific primary cells or cell lines that are related to CNS and CSF which are used to generated MPs in cell culture, controlled conditions (FIG. 4).

For example, the ability of various CNS cell populations to release MPs in vitro can be evaluated in response to various proapototic stimuli, such as H₂O₂ or Staurosporine, using a neuroblastoma cell line (SH-SY5Y), an in vitro system that has been used for evaluating the neuroprotective efficacy of compounds such as PPARdelta agonists (Iwashita A et al., 2007) or muscarinic receptors (De Sarno P et al., 2003).

SH-SY5Y cells were allowed to differentiate in vitro for 10 days and then exposed to increasing concentrations of Staurosporine. The cell culture medium is collected and used for quantifying total MPs levels by flow cytometry. The treatment of differentiated SH-SY5Y cells with Staurosporine resulted in a significant and dose-dependent increase in Annexin V-positive MPs (FIG. 5A). Similar results were obtained using with cells H₂O₂ as a stimulus.

If the MPs that are obtained in the three conditions are compared qualitatively by two-dimensional gel electrophoresis, there is an evident enrichment in proteins that is related to the exposure to Staurosporine (FIG. 5B). For instance, if the intensity of spots that correspond to specific proteins in Box C gradually increases, the intensity of other spots localized in Box 1 and Box 2 increases much more strongly. This observation suggests that Staurosporine-induced MPs contain a specific set of proteins which can be identified and better characterized in other cell types and/or in other cell culture conditions for determining antigens that can be used for identifying specific CS-MPs subpopulations.

For example, cell lines of neuronal (such as SH-SY5Y) or glial (such as CCF-STTG1) origin have been exposed to an apoptotic stimulus (such as Staurosporine). These cells produce MPs that can be detected in the cell culture medium as presenting phosphatidylserine on their surface (due to Annexin V binding) and/or a CNS-associated cell surface antigen such as NCAM (neural cell adhesion molecule or CD56) that is involved in the formation of neural circuits by interacting with extracellular molecules and by providing specific signals intracellularly (Schmid R and Maness P, 2008). Another in vitro system for studying CS-MPs in connection to neuroinflammatory response can be based on the murine microglial cell line BV-2 (Blasi E et al., 1990) that has been used to test compounds such as PPARalpha agonists (Ramanan S et al., 2008) or agonists of metabotropic glutamate receptors (Loane D J et al., 2009). These cells can be stimulated in serum-free medium with increasing concentrations of LPS (0, 10, 100, 1000 ng/ml) in order to evaluate the increase in Annexin V-positive MPs and/or the alteration in their composition, in particular of cell surface antigens that can be then used for studying CS-MPs populations.

These results indicate that cell lines of neuronal and glial origin may be able to release MPs in response to specific stimuli, and that this populations of MPs can be used for studying the presence of cell-type specific antigens that are associated to CS-MPs, and consequently CS-MPs subpopulations that can be of even higher interest for evaluating the status of a CNS disorder or a drug treatment. Extensive lists of antigens associated to CNS-associated cell types or tissues, in particular of neuronal origin that can contribute to CSF composition have been generated by different means in the literature (FIG. 6). By applying this approach to a panel of CNS-derived cell lines and a panel of antibodies that recognize CNS-associated antigen, specific antibodies can be chosen for selecting CS-MPs subpopulations and studying their variability in CSF samples that have been isolated from animals or patients. Antibodies or other molecules that specifically bind such antigens can be used for detecting MPs subpopulations within the CSF and/or in cell culture, and evaluating the variation in the amount and the molecular features of such MPs subpopulations in connection to a CNS disorder, a drug treatment, or any other condition.

Taken together, those results suggest that CS-MPs populations can be used for defining and using novel biomarkers that reflecting the pathophysiological status of the CNS in clinical practice and drug discovery and development. CS-MPs can be used for d prediction, diagnosis, prognosis and follow-up of CNS disorders with a strong focus on neurodegenerative disorders such as Parkinson, Alzheimer and multiple sclerosis. The presence of CS-MPs can be evaluated not only by classical immunological technology but also by measuring the procoagulant activity in the sample, or by detecting in electronic or atomic force microscopy.

### Example 3

### Analysis of MP Signature in Human CSF Samples

### Materials & Methods

### Isolation and Quantification of Human CS-MPs

The CSF samples were taken for diagnostic purpose from adult patients in medical institution by a lumbar puncture according to a standard procedure. Scientific use of CSF samples was approved by the local Ethics Committee and all patients gave a written informed consent for the diagnostic procedure. No traumatic signs were detected in all the patients.

50 to 300 μL of CSF was collected from each subject. CS-MPs were obtained after two sequential centrifugations. first at 1,500 g for 15 minutes at room temperature. The supernatant was then carefully removed and transferred to a new tube. The second centrifugation was performed at 13,000 g for 2 minutes at room temperature. Again, the supernatant was carefully transferred into a new tube and snap-frozen using liquid nitrogen. CS-MPs were stored at −80° C. until use.

CS-MPs quantifications were performed using fluorescein-labelled Annexin V and flow cytometry, as indicated in Example 1 and 2.

### Results & Conclusions

Human CS-MPs can be identified and characterized in patients that have been selected by different criteria (for example at risk, suffering, or under treatment for a CNS disorder). The previous examples have demonstrated that, by combining in vitro and in vivo studies that CNS-derived cell populations such as neuronal cells are able to produce MPs in response to various stimuli and that can be detected and isolated from CSF as CS-MPs. The concentration of such MPs could be increased in response to an acute stress (such as the intrecerebroventricular injection of LPS in rat), suggesting that CS-MPs may be considered as novel biomarkers for CNS disorders.

The detection and the isolation of CS-MPs can be performed also in human CSF that is obtained by individuals at risk or suffering of a CNS disorder. Human CSF samples can be collected by lumbar puncture in the morning, centrifuged and stored at −80° C. in polypropylene tubes. Samples remained frozen until analysis with His-tagged, Fluorescein-labelled Annexin V by flow cytometry. The CS-MPs can be also processed for proteomics studies for identifying biomarkers of potential interest using technologies that are described in the literature (Hale J et al., 2008; Hwang H et al., 2010; Shi M et al., 2009; Roche S et al., 2008; Garcia B et al., 2005; Jin M et al., 2005).

This approach has been tested by selecting the ten CSF samples from human subjects. Nine samples were obtained from different control subjects that were diagnosed, according to clinical scores, for a mild cognitive disorder and initial signs of dementia. The remaining sample was obtained from a test subject that was diagnosed, according to clinical scores, for a huge cognitive disorder according to clinical scores, and evident signs of dementia. CS-MPs levels are consistently low but detectable (12±3.8 MPs/μl) in all the nine control subjects but the CS-MPs level was increased more than 4 fold higher (53 MPs/μL) in the test subject (FIG. 7). The magnitude of this increase correlates well with both the significant 3-fold induction of CS-MPs levels observed in LPS injected rats (FIG. 3A), and the significant increase of MPs levels in Staurosporine-induced SH-SY5Y cells (FIG. 5A). Taken together, those results suggest that CS-MPs can be used for identifying biomarkers that are associated to clinical status and the progression of CNS disorders.

### REFERENCES

Bachy I et al., 2008. Brain Res Bull 75, 289-294.
Bauer B et al., 2005. Exp Biol Med. 230: 118-127.
Bianco F et al., 2005. J. Immunol. 174: 7268-7277.
Bianco F et al., 2009. EMBO J. 28: 1043-1054.
Blasi E et al., 1990. J Neuroimmunol. 27: 229-37.
Boesenberg-Grosse C et al. 2006. BMC Neurol. 6:35.
Burnier L et al., 2009. Thromb Haemost, 101(3):439-51.
Cayrol R et al., 2008. Nat Immunol. 9:137-45.
Combes V et al., 2005. Am J Pathol 166: 295-302.
De Jong, D et al., 2007. Clin Chem Lab Med. 45: 1421-1434
De Sarno Petal., 2003. J Biol Chem. 278:11086-93.
Dean C and Dresbach T, 2006. Trends Neurosci. 29:21-9.
Doeuvre L et al., 2009. J Neurochem. 110: 457-68.
Dubois B et al., 2007. Lancet Neurol 6, 734-746.
Faille D et al., 2009. FASEB J. 23: 3449-58. [Epub 2009 Jun. 17]
Finehout E et al., 2007. Ann Neurol 61: 120-129.
Fonteh A et al., 2006. Dis Markers 22:; 39-64.
Garcia B et al., 2005. J Proteom Res. 4: 1516-1521.
Hale J et al., 2008. Methods Mol Biol. 425:53-66.
Hampel H et al., 2008. Alzheimers Dement. 4: 38-48.
Hawkins B and Davis T, 2005. Pharmacol Rev. 57: 173-85.
Heegaard N et al., 2008. HUPO 2008 abstract.
Horstman L et al., 2007. Int Rev Neurobiol. 79: 227-68.
Hu S et al., 2006. Proteomics. 6: 6326-6353.
Hu Y et al., 2005. Mol Cell Proteomics. 4: 2000-9
Huang J et al., 2007. J Sep Sci. 30:214-225.
Huang M et al., 2009. Surg Neurol, 72:481-9. [Epub 2009 Mar. 27]
Huttner H et al., 2008. Stem Cells 26, 698-705.
Hwang H et al., 2010. Mass Spectrom Rev. 29: 79-125. [Epub 2009 Apr. 8]
Iwashita A et al., 2007. J Pharmacol Exp Ther. 320(3):1087-96
Jenner P, 2008. Ann Neurol. 64 (Suppl 2):S16-29.
Jin M et al., 2005. Proteomics. 5: 1940-52.
Johanson C et al., 2008. Cerebrospinal Fluid Res. 5: 10.
Jung D et al., 2005. J Immunol, 174: 6467-6.
Kong G et al., 2002. Glia. 37: 191-205.
Laterza O et al., 2006. Clin Chem. 52:1713-21.
Lehmensiek V at al., 2007. Mult Scler 13, 840-849.
Lemmon M, 2008. Nat Rev Mol Cell Biol. 9: 99-111.
Li S et al., 2009. Nucleic Acids Res. 37: D907-912.
Loane D J et al., 2009. J Biol Chem. 284:15629-39.
Lyck L et al., 2008. J Histochem Cytochem. 56:201-21.
Marzesco A et al., 2005. J Cell Sci. 118: 2849-2858.
Minagar A et al., 2001. Neurology. 56: 1319-1324.
Morel N et al., 2008 J Trauma 64:698-704.
Morel O et al., 2009. Haematologica. 94:313-317.
Nagano T et al., 2006. Glia. 53: 67-73.
Osumi K et al., 2001. Thromb Haemost. 85: 326-30
Pan S et al., 2007. Proteomics 7, 469-473.
Pardridge W, 2007. Stroke. 38 : 686-690.
Pasvogel A et al., 2008 Biol res Nurs. 10: 113-120.
Pritchard J, 2008. BMC Neurosci. 9(Suppl 3):S1.
Ramanan S et al., 2008. Free Radic Biol Med. 45:1695-704.
Roche S et al., 2008. Proteomics Clin Appl. 2:428-436.
Rohl T and Kurreck J, 2006. J Neurochem 99: 371-380.
Sanna P et al., 1995. Proc Natl Acad Sci USA, 92:272-5.
Sano Y et al., 2007. Cell Struct Funct. 32:139-47.
Schmid R and Maness P, 2008. Curr Opin Neurobiol. 18: 245-50.
Scolding N et al., 1989. Nature. 339: 620-2.
Shaw L et al., 2007. Nat Rev Drug Discov 6:295-303.
Shaw L et al., 2009. Ann Neurol. 65: 403-13.
Shi M et al., 2009. Neurobiol Dis. 35:157-64.
Smalheiser N, 2009. Cardiovasc Psychiatry Neurol, 2009:383086. [Epub 2009 Aug. 3]
Stace C and Ktistakis N, 2006. Bioch Biophys Acta. 1761: 913-926.
Steinmetz K and Spack E, 2009. BMC Neurol. 9 (Suppl 1): S2.
Stoica B et al., 2005. Mol Cell Neurosci 29, 355-371.
Thery C et al., 2009. Nat Rev Immunol. 9:581-93.
Thouvenot E et al., 2008. J Proteome Res. 7, 4409-4421.
Tumani H et al., 2008. Expert Rev Mol Diagn 8, 479-494.
Van Dreden P et al., 2009. Blood Coagul Fibrinolysis. 20: 494-502.
Visser P et al., 2009 Lancet Neurol 8:619-627
Yanagisawa M and Yu R, 2007. Glycobiol. 17: 57R-74R.
Zappaterra M et al., 2007. J Proteome Res 6, 3537-3548.
Zhang J et al., 2008. Am J Clin Pathol. 29: 526-9.
Zougman A et at., 2008. J Proteome Res 7, 386-399.
Zujovic V et al., 2001. J Neuroimmunol, 115: 135-43

1. A method for identifying Cerebrospinal Microparticles (CS-MPs) comprising the following steps:
a) Obtaining a Cerebrospinal Fluid (CSF) sample from a subject;
b) Isolating the acellular fraction of said CSF sample;
c) Separating the CS-MPs from the said acellular fraction by means of their dimension and the presence at its surface of at least one molecule that is known to be associated to cells forming the brain, the spinal cord, the Blood-Brain Barrier (BBB), the Blood-CSF Barrier (BCSFB), or the brain-CSF interface;
wherein the CS-MPs have a diameter comprised between 100 and 1000 nanometers and contain phosophatidylserine, and
wherein said molecule is selected from the group consisting of GAD67, SNAP25, Synaptobrevin 2, Neurogranin, Internexin, Zygin, NeuN, CD45, S100beta, MAP-2, GFAP, (Phospho)-Tau, ACE, Hemopexin, Transferrin, Attractin, Carbonic anydrase, IgG, Anti-MBP antibody, BDNF, IL-8, VDBP, 14-3-3, Neuron-Specific Enolase, NCAM (CD56), Neuroligins, neurexins, Glycoconjugates, ALCAM, BBB transporter proteins, Claudin-5 and Occludin.

2. The method of claim 1 wherein at least an agent having affinity for said molecule is used in step c).

3. The method of claim 1 wherein said molecule is NCAM which is known to be associated to neuronal cells.

4. The method of claim 1 wherein a phospholipid-binding agent is further used in step C.

5. The method of claim 2 wherein said agent is an antibody, a protein that binds a cell surface antigen, a peptide, or an inorganic compound.

6. The method of claim 5 wherein said agent is labeled and/or is immobilized on a solid phase.

7. CS-MPs obtained according to the method of claim 1.

8-12. (canceled)

13. A kit for isolating and/or using CS-MPs of claim 7 comprising
at least a phospholipid-binding agent used in step c and
an agent having affinity for molecules that are known to be associated to cells forming the brain, the spinal cord, the BBB, the BCSFB, or the brain-CSF interface wherein the CS-MPs have a diameter comprised between 100 and 1000 nanometers and contain phosophatidylserine, and wherein said molecule is selected from the group consisting of GAD67, SNAP25, Synaptobrevin 2, Neurogranin, Internexin, Zygin, NeuN, CD45, S100beta, MAP-2, GFAP, (Phospho)-Tau, ACE, Hemopexin, Transferrin, Attractin, Carbonic anydrase, IgG, Anti-MBP antibody, BDNF, IL-8, VDBP, 14-3-3, Neuron-Specific Enolase, NCAM (CD56), Neuroligins, neurexins, Glycoconjugates, ALCAM, BBB transporter proteins, Claudin-5 and Occludin.

14-16. (canceled)

17. An in vitro method for diagnosing or monitoring a CNS disorder in a patient that comprises the determination of the concentration and/or the composition of the CS-MPs of claim 7 in a Cerebrospinal Fluid sample from said subject.

18. (canceled)

19. The method of claim 4 wherein said phospholipid-binding agent is a phosphatidylserine-binding agent.

20. The method of claim 5 wherein said phosphatidylserine-binding agent is Annexin V.

21. The method of claim 1 wherein the CS-MPs are separated from the acellular fraction by means of their dimension by cytometry, microfiltration or centrifugation.
